(19) United States
(12) Patent Application Publication
(10) Pub. No.: US 2010/0137870 A1
(43) Pub. Date: Jun. 3, 2010

(54) ACETABULAR LINER INSERTER GUIDE

(75) Inventors: Jeffrey Joel Shea, Memphis, TN (US); William L. Waltersdorff, Hernando, MS (US); Charles Wayne Allen, Southaven, MS (US); Philip E. Frederick, Memphis, TN (US)

Correspondence Address: DIANA HOUSTON;SMITH & NEPHEW, INC., 1450 BROOKS ROAD, MEMPHIS, TN, 38116, US

(73) Assignee: SMITH & NEPHEW, INC., Memphis, TN (US)

(21) Appl. No.: 12/529,169

(22) PCT Filed: Feb. 28, 2008
(86) PCT No.: PCT/US2008/055323

§ 371 Date: Feb. 2, 2010

Related U.S. Application Data
(60) Provisional application No. 60/892,139, filed on Feb. 28, 2007.

(51) Int. Cl. A61B 17/56; A61F 2/32

(52) U.S. Cl. 606/91; 623/22.21

(57) ABSTRACT

An acetabular liner insertion guide (10) aligns a liner (12) within an acetabular shell. The liner includes a ring (16) and a penetrable layer (14). The ring includes a lip configured to rest on an upper surface of the acetabular shell. The ring is also configured to attach to the liner such that an upper surface of the liner is in a plane that is generally parallel to a plane that includes the upper surface of the acetabular shell. The penetrable layer is configured to receive an impactor and overlie the liner. When the insertion guide is placed on the shell and the impactor impacts the liner, the insertion guide separates from the liner and remains on the impactor.

### RELATED APPLICATIONS

This application claims priority to U.S. Provisional Application 60/892,139 filed Feb. 28, 2007.

### BACKGROUND

1. Field of the Related Art

The present application relates to prostheses, and more particularly relates to devices for inserting and impacting prostheses.

2. Related Art

Shells for hip prostheses may use a hard liner inserted into the shell. When inserting the hard liners, it is important to properly align the liner relative to the shell. Misalignment may create problems with micromotion of the liner relative to the shell. In addition, misalignment may create an uneven force distribution around the liner and may significantly reduce the potential for liner fracture.

CeramTec has developed an adapter that can be used with ceramic liners to ensure proper alignment with a shell before impacting the liner into the shell. However, this adapter is large and cumbersome. The adapter has three flex prongs at its periphery that grasp the edge of a liner at only a limited number of contact points along its periphery. U.S. Pat. No. 6,468,281 describes this method in more detail. A central thumb-activated plunger is used to impact the liner to the shell. Additionally, U.S. Pat. No. 5,169,399 and U.S. Pat. No. 5,571,111 show other alignment guides of the prior art which lack the inventive features of the present invention.

### SUMMARY

In one embodiment, a liner guide comprises a capture portion, a positioning portion, and a passageway. The capture portion is configured to capture a liner. The positioning portion is configured to overlie a shell. The positioning portion is configured to concentrically position and rotationally center the liner within the shell. The passageway is configured to receive an impactor such that when the impactor is received through the passageway and impacted. The capture portion releases the liner and the positioning portion directs the liner into the shell.

Alternatively, another embodiment includes a shell implanted in an acetabulum.

Another embodiment includes a capture portion having an upper portion and a lip portion. The liner has a diameter. The upper portion is configured to overlie the liner. The upper portion has a diameter approximately equal to the diameter of the liner. The lip portion is generally perpendicular to the upper portion and extends from the outermost edge of the upper portion. The lip portion exerts a generally radially oriented force against a side of the liner.

An alternative embodiment includes a capture portion comprising an upper portion configured to overlie the liner. A first lip portion is generally perpendicular to the upper portion and extends from the outermost edge of the upper portion. A second lip portion is generally perpendicular to the upper portion and extends from an innermost edge of the upper portion, the lip portions exerting clamping force against the liner.

Another embodiment includes a positioning portion that includes a flat flange extending perpendicularly from the capture portion.

Alternatively, the flat flange extends completely around the capture.

In another embodiment, the liner includes a passageway that includes a first diameter generally equal to the inner diameter of the liner and projections extending inward from the first diameter such that the innermost portions of the inward projections defines a second diameter less than the first diameter and greater than the diameter of an impactor head.

Alternatively, the inward projections are intermittently projected from the capture portion.

Another embodiment includes a method of inserting a liner into a shell. A step captures a liner within a liner guide. A step positions the liner within the shell such that the liner is concentrically and rotationally centered. Another step extends an impactor through the liner guide. A step impacts the impactor.

An alternative method further comprises the step of retaining the liner guide on the impactor.

Additionally, the shell may be implanted in an acetabulum.

Alternatively, the capturing step may comprise exerting a radially force around the liner guide against an outer surface of the liner.

An alternative method includes a capturing step that comprises exerting a clamping force between an inner and outer surface of the liner.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 is a perspective view of an embodiment of an insertion liner guide attached to a liner;

FIG. 2 is a partial perspective exploded view of the insertion liner guide and liner of FIG. 1;

FIG. 3 is a cross-section view of the insertion liner guide of FIG. 1;

FIG. 4 is a perspective view of another embodiment of an insertion liner guide;

FIG. 5 is a top view of the insertion liner guide of FIG. 4;

FIG. 6 is a top view of another embodiment of an insertion liner guide;

FIG. 7 is a perspective view of another embodiment of an insertion liner guide over an acetabular shell;

FIG. 8 is a perspective view of another embodiment of an insertion liner guide coupled to a liner;

FIG. 9 is a top view of the insertion liner guide of FIG. 8;

FIG. 10 is an example of implantation of a liner in an acetabulum;

FIGS. 11-14 are diagrams of an embodiment of the steps for implanting a liner in an acetabular shell;

FIGS. 15A and 15B are cross sectional views of an embodiment of an insertion liner guide attached to a liner and overlying a shell;

FIG. 16 is a top view of additional embodiments of an insertion liner guide; and

FIGS. 17-20 are cross-sectional partial side views of embodiments of insertion liner guides and liners.

### DETAILED DESCRIPTION OF THE EMBODIMENTS

The following description of the preferred embodiment(s) is merely exemplary in nature and is in no way intended to limit the invention, its application, or uses.

If a liner of an acetabular shell is inadvertently situated crooked, then there is a risk of fracturing the liner, deforming the liner, deforming the shell, and/or compromising the taper lock fit between the two during impaction. If any one of the abovementioned occurs, it is both time consuming to remove debris from the body cavity (in the case of fracture), and financially costly because a replacement liner implant and shell implant must be purchased and used. The surgical procedure takes longer than normal because the surgeon must ensure that all debris is removed (e.g., if a ceramic liner shatters). Such debris can cause significant “grinding” if any non-removed particles get between the new articulating surfaces or into surrounding bone and soft tissue.

Additionally, if the integrity of the liner or shell is compromised due to misalignment prior to impaction, the acetabular shell must be completely removed from the acetabulum. In other words, if there is any possibility that the metal taper in the shell may have deformed such that it may prevent a good taper lock, the surgeon will need to replace the shell as a conservative measure. If there is no identical replacement prosthesis readily available, compromises may need to be made.

The problem with this situation is that if good initial stability has been established, then the removal of the deformed shell from the bone to insert another is more invasive, risks poor stability for the second shell, and would cost double (due to the patient needing a total of two implants instead of one to complete the surgery).

Therefore, there is a need to ensure proper concentric orientation and axial alignment of a liner prior to impaction, in order to reduce: the cost of non-necessary replacement liners and shells, the risk of a lengthy and complicated procedure, and the risk of potential prosthesis failure in the future due to wear accelerated by residual debris or a dislocation caused by a failed taper lock.

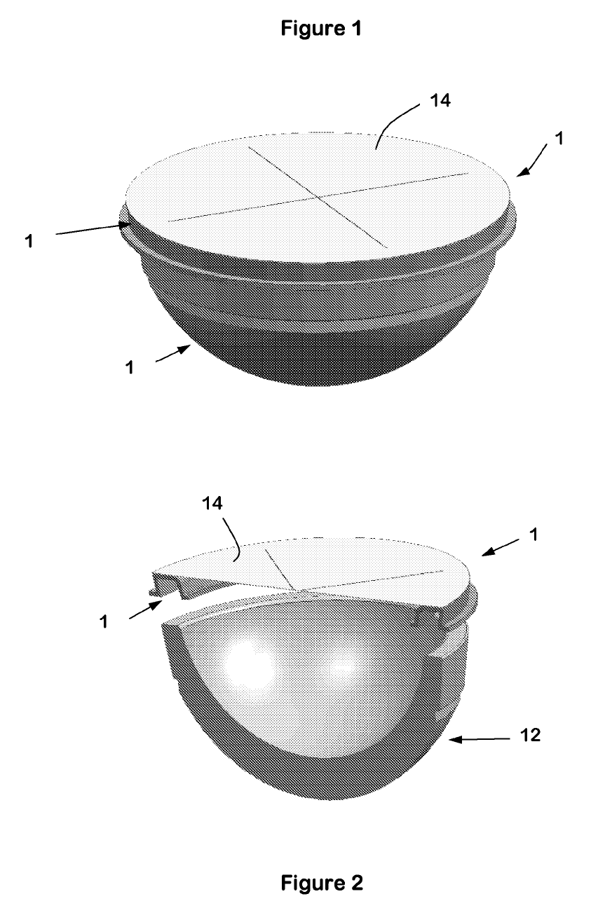

Turning now to the figures, FIG. 1 is a perspective view of an embodiment of an insertion liner guide 10 attached to a liner 12. The acetabular liner inserter guide 10 is used to assure alignment of the liner 12 in an acetabular shell. The guide 10 includes a lid 14 and a liner capture ring 16. The lid 14 may grab onto an impactor to minimize the risk of accidental implantation of a part of the liner inserter guide 10. A warning label may be a part of the lid 14 to warn a surgeon that the guide 10 is not meant for implantation. The capture ring 16 attaches to the lid 14 and the capture ring 16. The lid 14 may be slotted to allow an impactor to penetrate the lid 14 while maintaining the integrity of the lid 14.

The ring 16 may be made out of PETG (the same material as implant package trays). Such a material may allow the ring 16 to be disposable. The PETG ring 16 by itself, not attached to a lid 14, may accidentally be implanted (left in undetected). Therefore, the lid 14 featuring a die-cut “X” through the center is attached to the upper surface of the ring 16. In one embodiment, the lid 14 may be a Tyvek lid similar to the lids of the PETG implant package trays. The Tyvek lid 14 may be attached to the ring 16 in the typical manner that a Tyvek lid is fixed to a PETG implant package tray, namely a heat seal process.

The Tyvek lid 14 may be highly visible. For example, the lid 14 may be white. Such highly visible guides reduces the chances of accidental implantation. In addition, the Tyvek lid 14 may include markings such as a company logo or may include important instructions such as “Do Not Implant”, “Disposable Hard Bearing Inserter”, reassembly instructions, etc. Such instructions may be printed on the lid 14 in highly visible hues, both increasing the visibility of the guide 10 and the instructions. The die-cut “X” in the Tyvek lid 14 may hold the disposable ring/lid 16 assembly to the shaft of an impactor instrument so it is extracted from the body simultaneously with the impactor instrument. These simple, disposable guides 10 may be included in the packaging of the liner/bearing surface. In addition, additional guides may be slid into the original packaging to protect the sterile field such as would occur when one lid pre-assembled on the hard bearing is dropped on the floor.

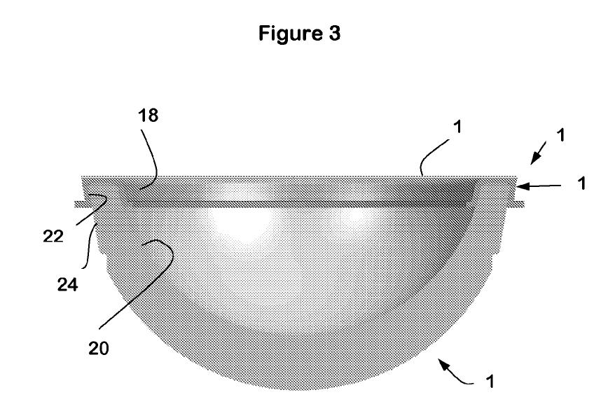

Turning now to FIG. 2, FIG. 2 is a partial perspective exploded view of the insertion liner guide 10 and liner 12 of FIG. 1. The capture ring 16 of the liner guide 10 may be configured to completely overlie an upper portion of the liner 12. The capture ring 16, then, may be placed over the liner 12 and snugly fit to the liner 12 such that the lid 14 may be generally parallel to an upper surface 17 of the liner 12.

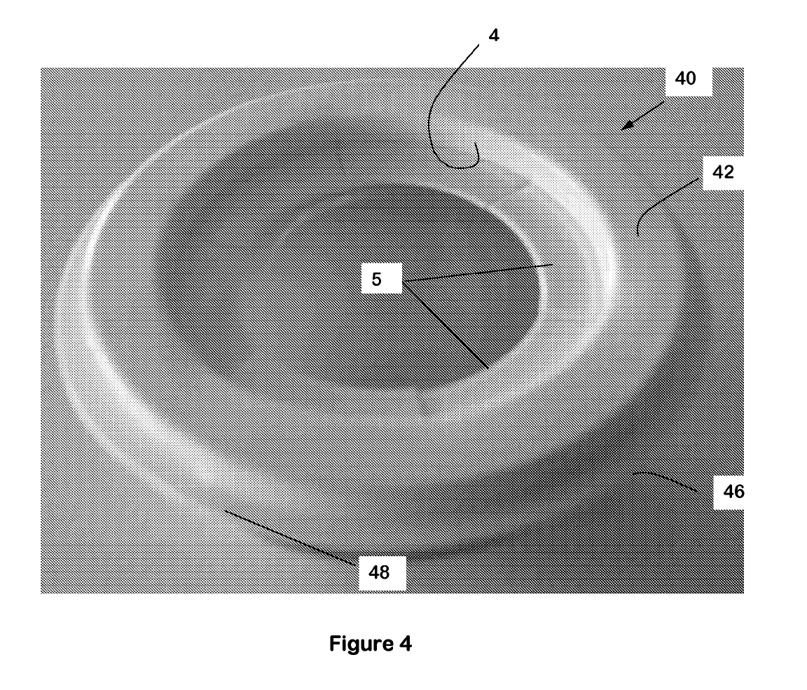

Turning now to FIG. 3, FIG. 3 is a cross-section view of the insertion liner guide 10 of FIG. 1. The capture ring 16 includes an inner ring surface 18 configured to abut an inner surface 20 of the liner 12 and an outer ring surface 22 configured to abut an outer surface 22 of the liner 12. The distance between the inner ring surface 18 and the outer ring surface 22 is slightly less than the thickness of the liner 12 so that the ring surfaces 18 and 22 may press against the liner 12 when the guide 10 is placed on the liner 12. The force from the surfaces 18 and 22 against the liner 12 hold the guide 10 to the liner 12.

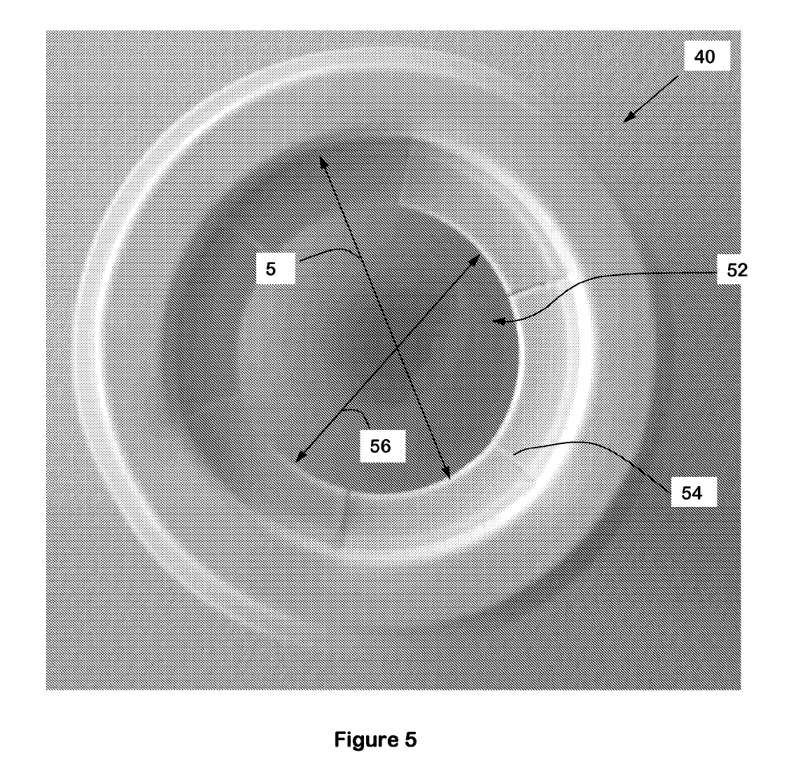

Turning now to FIG. 4, FIG. 4 is a perspective view of another embodiment of an insertion liner guide 40. The guide 40 comprises a ring 42 preferably made from a disposable plastics material and having at least some elastic properties. An inner lip 44 and outer lip 46 of the ring 42 extend generally parallel to each other and generally perpendicular to an upper surface of the ring 42. An outwardly projecting flange 48 and inward projections 50 are generally perpendicular to the lips 44 and 46 and generally parallel to the upper surface.

The ring 42 may be placed on the rim of a generally hemispherical acetabular liner, to cover a substantial portion or entire portion of the rim. The outwardly projecting flange 48 is configured to rest upon the liner. The inward projections 50 are configured to allow an impactor to be placed within the guide 40. The ring 42 is formed so as to be low in profile and have a tactile frictional engagement with the liner. The ring 42 is “snapped” to the liner's rim and is held in there by frictional forces.

The lips 44 and 46 are generally cylindrical; however, it may be interrupted or vary in shape so as to exhibit different spring properties and cause higher or lower frictional holding forces between the liner and the alignment guide 40. During insertion of the liner into the acetabular shell, the radially-outwardly extending flange 48 is configured to come in contact with the rim of an acetabular shell and act as a stop means. The combination of the downwardly-depending lip 46 and the radially-outwardly extending flange 48 provides and maintains a predetermined clearance between the liner and the acetabular shell (i.e., a “standoff”). The combination further serves as a soft tissue barrier which prevents overhanging tissue and bio matter from entering the taper lock interface surfaces.

The predetermined clearance held is preferably selected to be small enough to ensure that the liner and the acetabular shell are both concentrically and axially aligned, and also selected to ensure minimal relative movement between the liner and acetabular shell. However, the predetermined clearance is also preferably selected to be large enough such that the taper lock between the liner and shell does not fully engage. An impactor device having a shaft may extend through the ring 42 and impact the liner into the acetabular shell.

In a preferred embodiment, the internal diameter of the ring is provided with inward projections 50 as a means for capturing the ring 42 to the shaft of the impactor device. By capturing the ring 42 to the shaft of the impactor, the ring 42 does not become inadvertently lost inside the body cavity but instead remains fixed to the shaft of the impactor after impaction. The ring 42 is generally flexible enough to release the liner from its elastic grip during impaction and allow forces applied to the impactor to close the predetermined distance between the shell and liner and form a good taper lock. Once a good taper lock between the shell and liner is achieved, the impactor tool may be removed from the body cavity, with the alignment guide 40 still attached thereto. The projections for capturing the ring 42 to the impactor device retains and couples the alignment guide 40 to the shaft of the impactor tool until the alignment guide 40 is manually removed. The alignment guide 40 may be sterilized for a later second use, or may be properly disposed of.

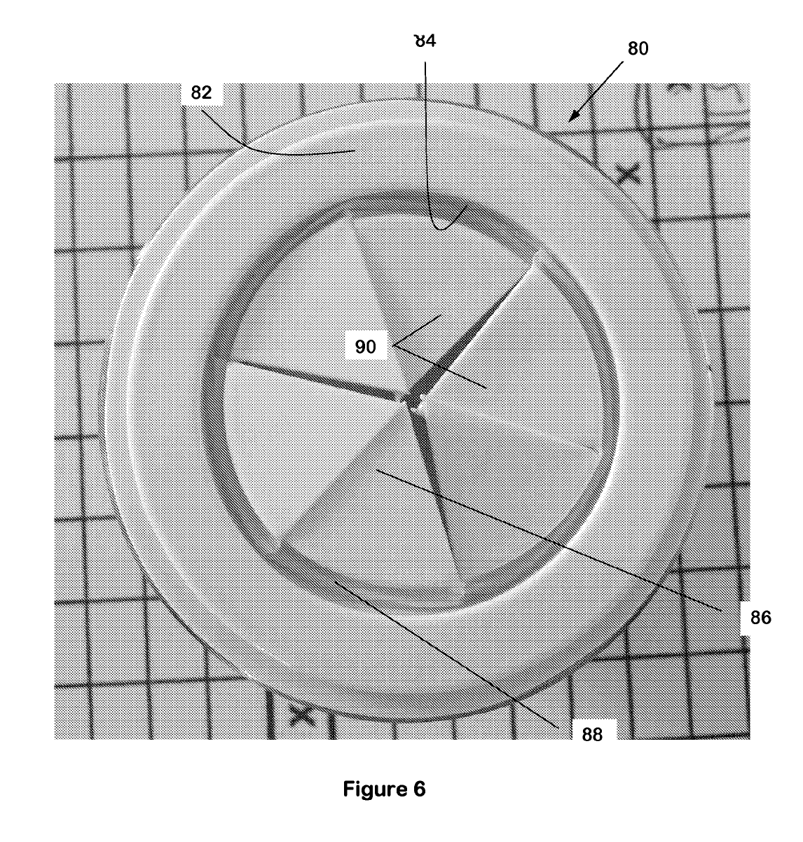

Turning now to FIG. 5, FIG. 5 is a top view of the insertion liner guide 40 of FIG. 4. The liner guide 40 includes an inner passageway 52 configured to receive an impactor. Projections 54 extend into the passageway 52. An inner diameter 56 of the passageway is narrower than the impactor. An outer diameter 58 of the passageway 52 is greater than the diameter of the impactor and approximately equal to the inner diameter of the liner. Thus, when the impactor is placed within the guide 40, the projections 54 extend over the impactor head.

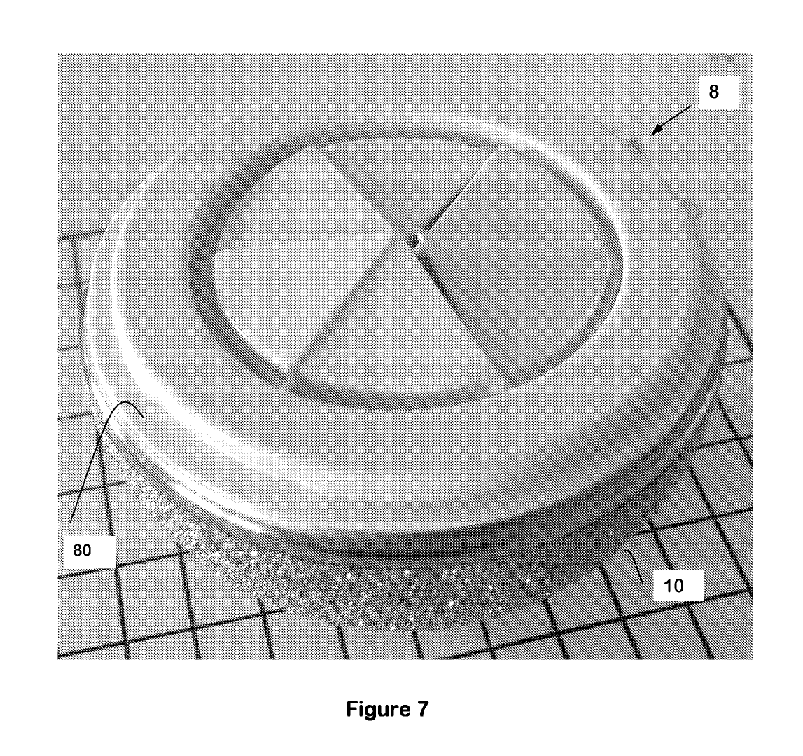

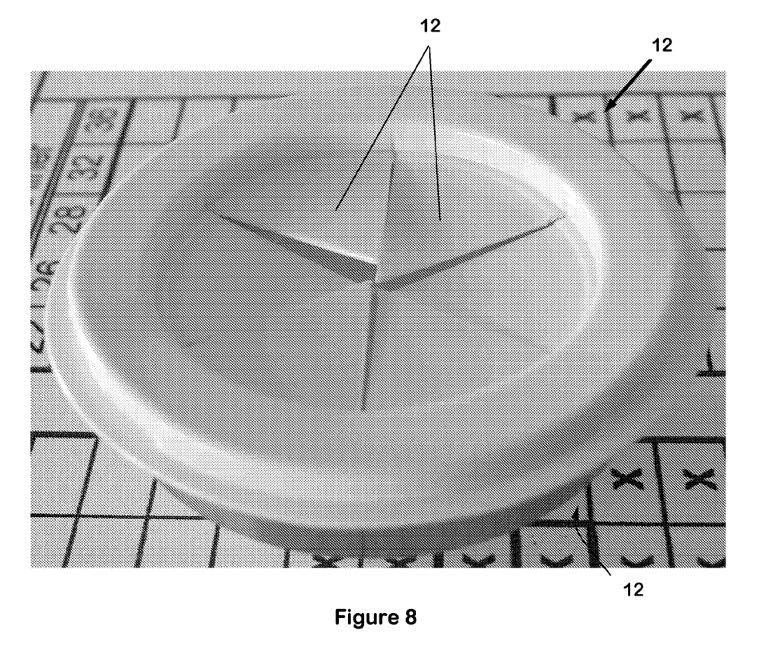

Turning now to FIGS. 6 and 7, FIG. 6 is a top view of another embodiment of an insertion liner guide 80. FIG. 7 is a perspective view of another embodiment of the insertion liner guide 80 over an acetabular shell 100. The guide 80 comprises a ring 82 preferably made from a disposable plastics material and having at least some elastic properties. An inner lip 84 and outer lip 86 of the ring 82 extend generally parallel to each other and generally perpendicular to an upper surface of the ring 82. An outwardly projecting flange 88 and inward projections 90 are generally perpendicular to the lips 44 and 46 and generally parallel to the upper surface. As shown in FIG. 7, the flange 88 of the guide 80 overlies the shell 100 to rest the liner within the shell such that a top portion of the shell is parallel with the top portion of the liner.

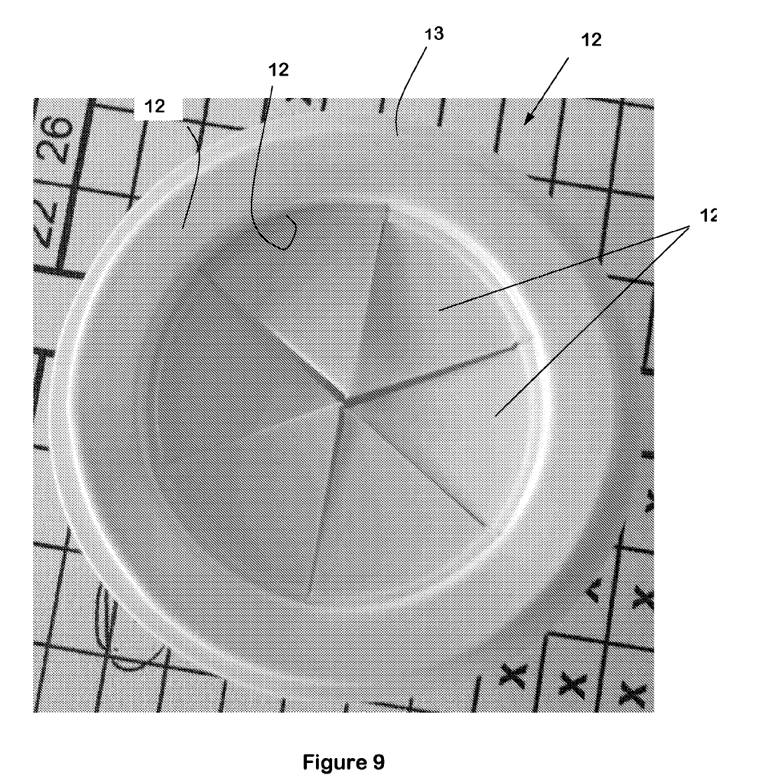

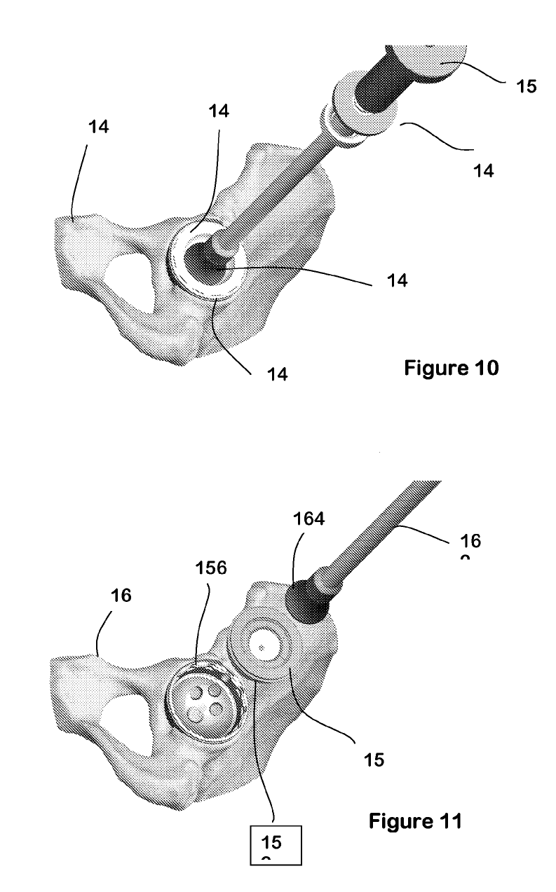

Turning now to FIGS. 8 and 9, FIG. 8 is a perspective view of another embodiment of an insertion liner guide 120 coupled to a liner. FIG. 9 is a top view of the insertion liner guide 120 of FIG. 8. In contrast to the guide 80 of FIGS. 6 and 7, projections 124 are recessed within the guide 120. The recessed projections 124 may provide a lower profile embodiment over a liner 122. The guide 120 comprises a ring 126 preferably made from a disposable plastics material and having at least some elastic properties. An inner lip 128 and outer lip 1306 of the ring 126 extend generally parallel to each other and generally perpendicular to an upper surface of the ring 82.

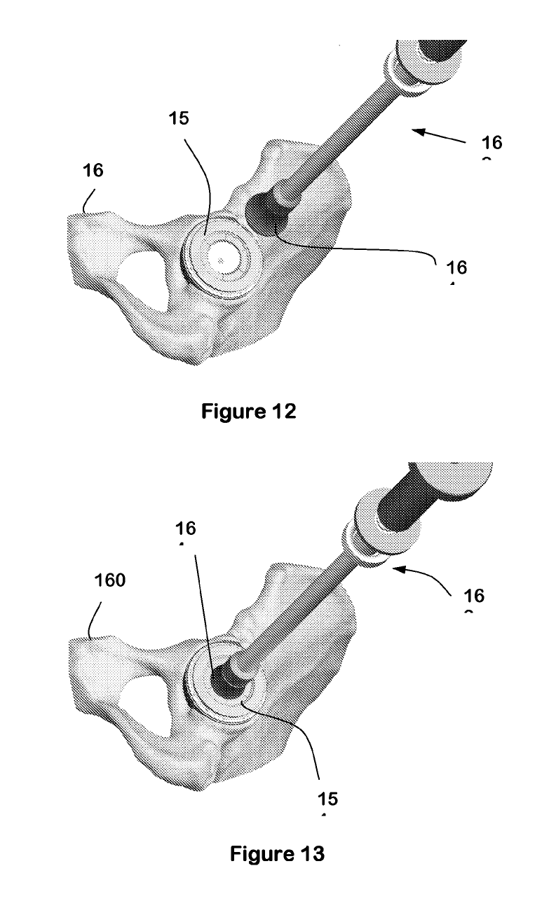

Turning now to FIG. 10, FIG. 10 is an example of implantation of a liner 140 in an acetabulum 144. The liner is impacted into a shell 142 implanted in the acetabulum. An impactor 146 includes an impactor head 148 and an impact face 150. The impact face 150 receives a blow to transmit the force through the impactor 146 and into the impactor head 148.

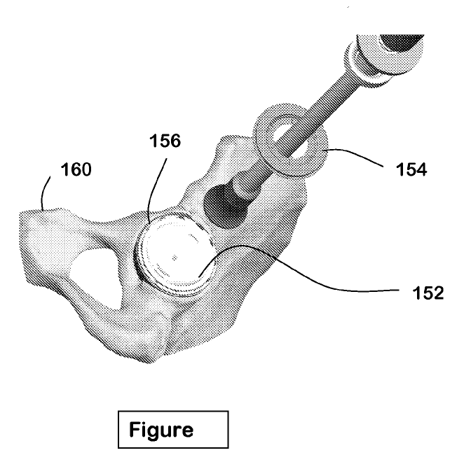

FIGS. 11-14 are diagrams of an embodiment of the steps for implanting a liner 152 in an acetabular shell 156 implanted in an acetabulum 160.

The alignment guide 152 is attached to the rim of a liner 152 (if not already assembled prior). An impactor 162 may be inserted through the center of the alignment guide 154 so that the impactor head 164 is within the liner 152. Engaging means formed on the internal diameter of the alignment guide allows the temporary passage of the ball/head end 164 of the impactor 162.

The engaging means on the inside diameter of the alignment guide 154 holds the liner 152 to the impactor 162 as one piece. The liner 152 is lowered into the acetabular shell 156 via the impactor 162. Alternatively, the liner 152/alignment guide 154 combination may be inserted by hand into the shell 156 first, and then once the combination is correctly situated within the acetabular shell 156, the impactor 162 can then be inserted through the alignment guide to finish assembly (as shown in FIGS. 11-14).

The outer circumferential downwardly-depending lip of the alignment guide 154 centers the liner on the acetabular shell along the rim of the shell (FIG. 12). The liner is spaced from the shell only so far as to prevent a taper lock between the liner and acetabular shell, and provide an otherwise very close spacing needed for good concentric and axial alignment. The radially-outwardly extending flange serves to form a planar contact surface with the rim of the acetabular shell and improve concentricity and axial alignment.

A force is applied to the impactor 162. Flexible portions of the alignment guide 154 having elastic properties which hold itself to the liner start to deform under stress. The engaging means on the inside diameter of the alignment guide 154 may or may not deform slightly under the force applied to the impactor 162.

Eventually, the force applied to the liner 154 through the impactor 162 overcomes the frictional holding forces between the alignment guide 154 and the liner. The liner slips out of the elastic holding portion of the alignment guide 154, and the forces applied to the impactor 162 push the liner into the acetabular shell.

The liner is “snap-locked” into the acetabular shell with a good taper lock. The energy stored in the alignment guide from flexion and distortion during insertion is released. The alignment guide 154 springs upward, and is guided by the shaft of the impactor 162 (FIG. 14).

Since the alignment guide 154 is formed with a ring shape, the shaft of the impactor 162 prevents the alignment guide from being displaced from the impactor 162. This ensures that the small alignment guide 154 is not accidentally left within a small body cavity, which is full of blood.

The alignment guide 154 finally loses all of the energy retained from elastic deformation during the impaction process. The outer diameter of the ball/head portion 164 of the impactor 162 is greater than the internal diameter of the alignment guide 154. Therefore, the alignment guide 154 is kept on the shaft of the impactor 162 and is not lost within the body cavity. The engaging means on the internal diameter of the alignment guide 154 preferably comprises spring fingers which flex to allow the insertion of the ball/head portion 164 of the impactor 162, but prevent the alignment guide 154 from inadvertently separating from the impactor 162 after impaction.

The alignment guide 154 and impactor 162 may be removed from the body cavity together, leaving the liner 152 properly aligned and fixed within the acetabular shell 156 (FIG. 14).

The alignment guide 152 may be removed from the impactor 162 by pulling it off of the shaft and over the ball/head portion 164. The engaging means on the internal diameter of the alignment guide 154 (e.g., spring fingers) flex to allow passage of the larger ball/head portion 164 of the impactor 162.

Once removed, the alignment guide 154 is then sterilized or properly disposed of. The liner 152 is correctly installed without worry of misimplantation or compromised taper locks.

The guides may be pre-assembled with a liner (before or after package sterilization) or not pre-assembled with a liner. For instance, the alignment guide may be packaged with a liner by the liner manufacturer, or may otherwise be packaged in a sterile manner by itself and given its own part number. It would be expected that the alignment guide of the present invention will have many various shapes and geometries to accommodate different liner sizes, and so an assorted collection of alignment guides having different sizes or configurations may be packaged together in a sterile manner. The assorted alignment guides may be individually wrapped and placed into a box, or the may all be placed into a single wrap and then placed into a box.

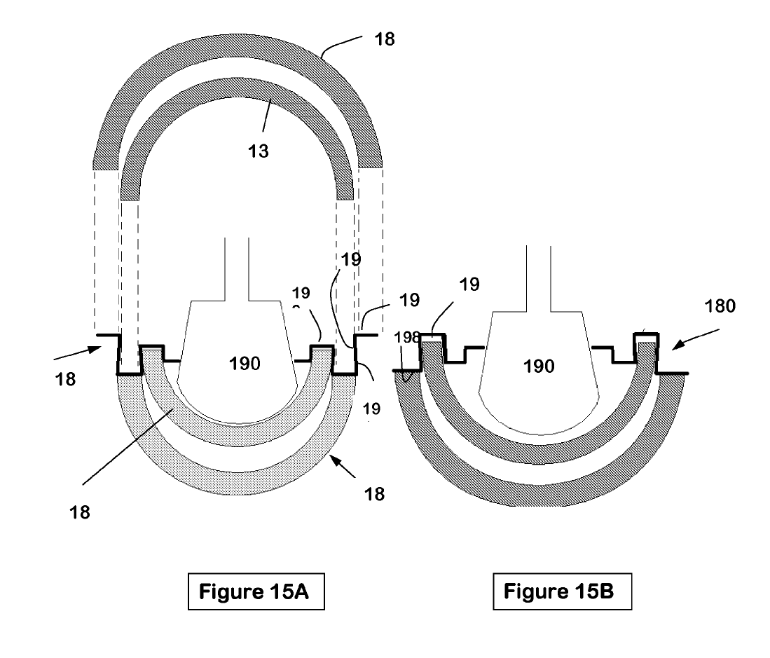

Turning now to FIGS. 15A and 15B, FIGS. 15A and 15B are cross sectional views of an embodiment of an insertion liner guide 180 attached to a liner 184 and overlying a shell 182. To consolidate the number of alignment guides used for a range of liner sizes, the alignment guide may be made reversible so as to accommodate at least two differently-sized liners and shells. By forming multiple alignment guide sizes into a single “universal” alignment guide, the number of pieces per kit is reduced, the tooling and mould costs are reduced, and the total cost for manufacturing and inventory is reduced due to higher quantities. The exact number of liners that may be used with a single alignment guide is limited only by the size of the incision and the possibility of interference with surrounding soft tissue.

As shown in FIG. 15A, a first orientation of the liner guide 180 grips the first liner 184 in a first fold 192. A flat portion 194 of the liner guide 180 overlies the first shell 182. An impactor 190 may impact the first liner 184 into the shell 182. The liner guide 180 may also be used with a second liner 188 and shell 186. The liner 188 is placed within a second fold 196 (as shown in FIG. 15B). The second fold 196 in this embodiment is opposite the first flat 194, but alternatively may be radially displaced from the first fold 192 and first flat 194. A second flat 198 overlies the second shell 186.

The alignment guide may be formed integrally with sterile packaging (e.g., molded into a container with perforations to remove it therefrom). The method of packaging an alignment guide does not affect the scope of the present invention. The alignment guide of the present invention may be advantageously used in combination with liners made of any material known in the art. For instance, liners made of polyethylene, metals, ceramics, or other conventional materials will work equally well with the present invention. The present invention may be a sterilizable permanent fixture to be included in a surgical tool kit, or it may be a disposable, or semi-permanent item.

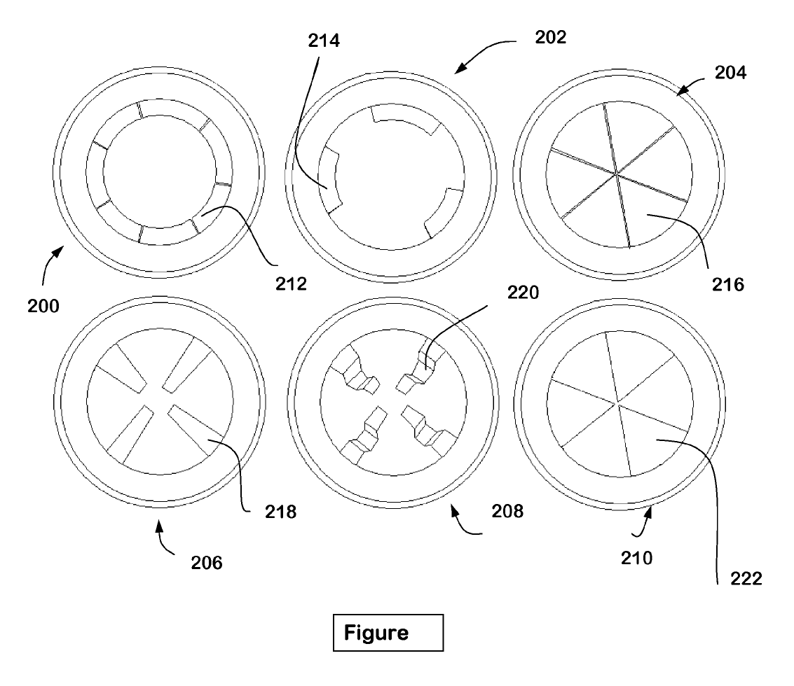

The guide preferably covers a total inner and outer circumferential portion of the rim of the liner for tight fitting. Additional examples of some guides are shown in FIG. 16. Liner guides 200-210 include inward projections 212-222 which are configured to retain the impactor within the ring. These embodiments may include an alignment guide formed as a split ring. An alignment guide having an interrupted engagement surface (such as the projections 214, 218, 220, and 222) so as to form intermittent contact points around the inner. Similarly, intermittent forms may be placed on the outer rim of any one of the liner and shell (i.e., for material savings or design indicia). The length of the projections may extend fully to the middle as the projections 216 and 222, have shallow projections like projections 212 and 214, or have an intermediate length like projections 218 and 220. Additionally, the projections may include folded portions such as the projections 220. The folded portions may absorb some of the forces of impaction.

Other ring geometries such as polygons (e.g., octagon) may be employed so long as the alignment guide is adequately configured for temporary attachment to the rim of a liner and does not interfere with soft tissue and bone. The alignment guide may or may not employ a textured surface or other tactile features such as bumps, ridges, or protrusions to provide additional gripping surfaces and/or to vary the flexibility characteristics of the ring (e.g., circular accordion-type ridges). Such tactile features may also be used to increase or decrease the friction between the liner and alignment guide, and may be practical in compensating for large tolerances in liner dimensions.

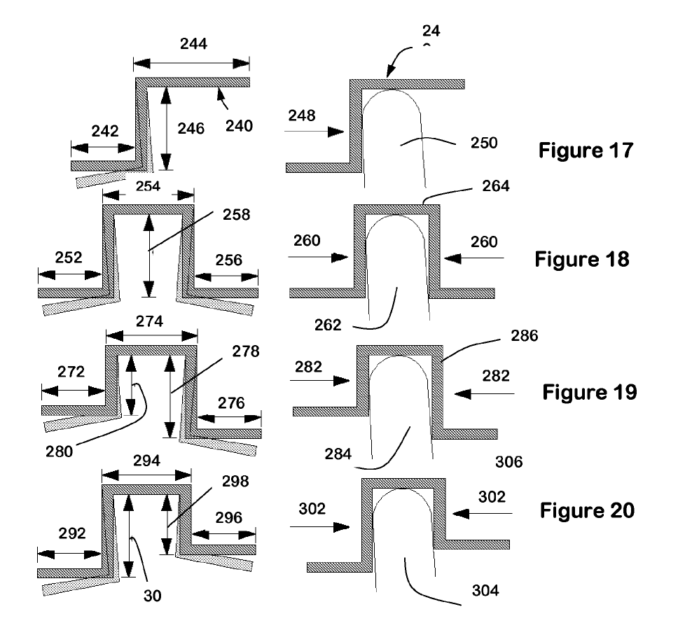

Turning now to FIGS. 17-20, FIGS. 17-20 are cross-sectional partial side views of embodiments of insertion liner guides and liners. Liner guides 240, 264, 286, and 306 are attached to liners 250, 262, 284 and 304. FIG. 17 depicts a profile shape where a radial force 248 pointing inward is applied between the liner guide 240 and liner 250. FIG. 18 depicts a profile shape where a pinching force 260 is symmetrically applied between the liner guide 264 and liner 260. FIG. 19 depicts a profile shape where a pinching force 282 is asymmetrically applied between the liner guide 286 and liner 284. FIG. 20 depicts a profile shape where a pinching force 302 is asymmetrically applied between the liner guide 306 and liner 304. The lengths 242, 252, 272, and 292 of the flanges can effect the amount of flexion of the guides 240, 264, 286, and 306 when the liner is impacted. Longer flanges would make the liners 240, 264, 286, and 306 flex less. Similarly, the depths 246 and 258 may also affect the amount of flexion in the liners 240 and 264. In addition, the depths of the guides 240, 264, 286, and 306 may also effect the profile of the liners and guides. A deeper depth may also limit the relative position of the impactor to the liner, as deeper liner guides allow for less movement of the impactor head under the liner guide. The widths 254, 274 and 294 of the top portions are sized to be generally equal to the width of the liner. The width 244 of the radial force only embodiment of FIG. 17 is restricted such that the diametrically opposite portion of the liner guide 240 should be a distance generally equal to the diameter of the liner away.

The asymmetric lengths 278, 280 and 298, 300 of the asymmetric profiles effect the center of the clamping forces 282 and 302. The asymmetric forces may effect how stable the liner guide attaches to the liner. For example, a liner guide that does not pop off may be adjusted by adjusting the asymmetry. Alternatively, a liner guide that pops off of the liner before impaction may also be adjusted by adjusting the asymmetry.

The present invention may be formed as an assembly of two or more separate pieces which are made integral (e.g., using heat fusion or adhesion means). Alternatively, the assembly may be formed of a single unitary material such as a homogeneously-moulded ABS or other cheap, preferably biocompatible plastic. The internal engagement means of the present invention may comprise a flexible inner lip, or any number of flexible finger members so as to retain the ring inserter to the head impactor before, during, and after impaction.

The present invention may be utilized with any modular portion of a hip or shoulder shell, the portion being of any material or geometry, in situations where proper insertion and alignment are critical. Such modular portions may include but are not limited to: liners, lockrings, and adapters. The present invention may also be used in non-medical applications for joining two cups which may or may not be at least partially spherical.

While the present invention is particularly useful with ceramic liners, it would be equally advantageous to use it with liners of various materials including cobalt chrome, oxidized zirconium, and others as discussed above.

Indicia of sorts may be incorporated into the alignment guide at various locations. The indicia may comprise corporate logos, trademarks, sizing info, material info, date of manufacture, instructions, warnings, etc.

The present invention may be incorporated into trial liners in order to make insertion and trial reduction easier. Such trial liners may be adapted for bipolar, tripolar, or multiple articulating joint prostheses. Trial liners may be formed integrally with the alignment guide of the present invention in the form of a co-moulded flange or the like. Alternatively, the present invention may be made integral with a trial liner via a threaded, snap-fit, or other connection feature known in the art.

The present invention may further be incorporated into the impactor tool, itself. Such an integration may be made in many different ways. If the alignment guide is made of a more rigid material, ball detents may keep it attached to the impactor and facilitate release during impaction. Alternatively, the impactor tool may be formed integrally with the flexible alignment guide of the present invention in the form of a flange or the like. As an alternative, the present invention may be made integral with the impactor tool via a threaded or other connection feature known in the art. If a normal femoral ball head is used for impaction, such a ball head may incorporate the annular alignment guide of the present invention in a similar manner as discussed above. The ball head may then be attached to an impactor shank via a Morse taper, thread, or the like.

The present invention ensures that a surgeon has correctly aligned and oriented a liner with an acetabular shell prior to impaction. Correct alignment is critical, because if a liner (in particular, ceramic) is misaligned and is then impacted, fracture is almost inevitable. Cleaning a body cavity of small fragments can be a very stressful and time-consuming process. Furthermore, there is no guarantee that all fragments are completely removed, and any remaining pieces will rapidly grind the prosthesis, surrounding soft tissue, and bone.

In the case of more robust plastic and metallic liners, misalignment prior to impaction may compromise the designed taper-lock fit between the two components, often requiring the removal of the acetabular shell from the bone, and insertion of a new replacement shell and liner. Any burr or deformation accidentally formed in either part may cause the insert to separate from the shell in-situ and lead to failure.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A liner guide, comprising:
a capture portion configured to capture a liner;
a positioning portion configured to overlie a shell, the positioning portion configured to concentrically position and rotationally center the liner within the shell; and
a passageway configured to receive an impactor through the liner guide such that when the impactor is received through the passageway and impacted, the capture portion releases the liner and the positioning portion directs the liner into the shell.

2. The liner guide of claim 1, wherein the shell is implanted in an acetabulum.

3. The liner guide of claim 1, wherein the liner has a diameter, and wherein the capture portion comprises: an upper portion configured to overlie the liner, the upper portion having a diameter approximately equal to the diameter of the liner; and a lip portion generally perpendicular to the upper portion and extending from the outermost edge of the upper portion, the lip portion exerts a generally radially oriented force against a side of the liner.

4. The liner of claim 1, wherein the capture portion comprises: an upper portion configured to overlie the liner; a first lip portion generally perpendicular to the upper portion and extending from the outermost edge of the upper portion; and a second lip portion generally perpendicular to the upper portion and extending from an innermost edge of the upper portion, the lip portions exerting clamping force against the liner.

5. The liner of claim 1, wherein the positioning portion includes a flat flange extending perpendicularly from the capture portion.

6. The liner of claim 5, wherein the flat flange extends completely around the capture portion.

7. The liner of claim 1, wherein the passageway includes a first diameter generally equal to the inner diameter of the liner and projections extending inward from the first diameter such that the innermost portions of the inward projections define a second diameter less than the first diameter and greater than the diameter of an impactor head.

8. The liner of claim 7, wherein the inward projections are intermittently projected from the capture portion.

9. A method of inserting a liner into a shell, comprising the steps of:
a. capturing a liner within a liner guide;
b. positioning the liner within the shell such that the liner is concentrically and rotationally centered;
c. passing an impactor through the liner guide captured on the liner; and
d. impacting the impactor.

10. The method of claim 9, further comprising the step of retaining the liner guide on the impactor.

11. The method of claim 9, wherein the shell is implanted in an acetabulum.

12. The method of claim 9, wherein the capturing step comprises exerting a radially force around the liner guide against an outer surface of the liner.

13. The method of claim 9, wherein the capturing step comprises exerting a clamping force between an inner and outer surface of the liner.
